(19) United States
(12) Patent Application Publication
(10) Pub. No.: US 2017/0175191 A1
(43) Pub. Date: Jun. 22, 2017

(54) METHOD FOR THE DIAGNOSIS AND/OR PROGNOSIS OF ACUTE RENAL DAMAGE

(71) Applicant: Fundación Para La Investigación Biomédica del Hospital Universitario Ramon y Cajal, Madrid (ES)

(72) Inventors: Elia Aguado Fraile, Madrid (ES); Miren Edurne Ramos Munoz, Madrid (ES); Angel Manuel Candela Toha, Madrid (ES); Fernando Liano Garcia, Madrid (ES); María Laura Garcia Bermejo, Madrid (ES)

(21) Appl. No.: 15/277,734

(22) Filed: Sep. 27, 2016

Related U.S. Application Data
(63) Continuation of application No. 14/364,644, filed on Jun. 11, 2014, now abandoned.

(30) Foreign Application Priority Data
Dec. 15, 2011 (ES) P 201132023

(51) Int. Cl. C12Q 1/68

(52) U.S. Cl. CPC C12Q 1/6883; C12Q 2600/178; C12Q 2600/158; C12Q 2600/118

(57) ABSTRACT

The invention relates to miRNAs: miR-26b, miR-29a, miR-454, miR-146a, miR-27a, mi-R93 and miR-10a, as markers of acute renal damage, and to a method and kit for the diagnosis and/or prognosis of acute renal damage using said markers.

### FIELD OF THE INVENTION

The present invention is encompassed in the general field of biomedicine and relates particularly to a method for the diagnosis and/or prognosis of acute renal damage.

### STATE OF THE ART

miRNAs are small, endogenously encoded RNAs (22-25 nucleotides) capable of recognizing messenger RNAs and thus negatively regulating protein expression within RNA-induced silencing complexes (RISCs) due to partial or complete complementarity with their target mRNA. Most miRNAs are transcribed by RNA Pol II from individual genes or from polycistronic transcripts for several of them at one time. They are generated as longer pre-miRs which are processed in the nucleus by ribonuclease III, move out to the cytoplasm by means of Exportin-5 and Ran-GTP-dependent mechanisms and are finally processed by another ribonuclease III into their mature form in the cytoplasm.

Their function is essential in a wide range of processes including embryonic development, stress response or strict regulation of physiological processes and, therefore, homeostasis maintenance of the body.

Very recently it has been demonstrated that miRNAs are key regulators in rapid and precise cell response to any type of stimulus including the lack of nutrients or hypoxia (Ivan M, Harris A L, Martelli F, Kulshreshtha R. Hypoxia response and microRNAs: no longer two separate worlds. J Cell Mol Med.; 12(5A):1426-31, 2008).

Patent application WO2011012074 describes a series of miRNAs including, among them, miR-29a, as plasma markers for liver cancer, and a method for the diagnosis and evaluation of liver cancer based on the detection of at least one of said miRNAs.

Patent application WO2009036236 relates to a series of miRNAs including, among them, miR-146a, as plasma markers for liver cancer, and a method for the diagnosis and/or evaluation of different oncologic pathologies based on the detection of at least one of the described microRNAs.

Akkina, S. et al., “MicroRNAs in renal function and disease”, Translational Research, 2011 April, Vol. 157, No. 4, pg 236-240 and Li, J. Y. et al., “Review: The role of microRNAs in renal disease”, NEPHROLOGY (CARLTON), September 2010, Vol. 15, No. 6, p 599-608, describe the involvement of miRNAs in renal physiology and pathology.

JUAN, D. et al., “Identification of a microRNA panel for clear-cell renal cancer”, UROLOGY, 2010 April, Vol. 75, No. 4, pages 835-41, describes a series of miRNAs as markers for renal cancer.

Acute renal failure (ARF) as a syndrome is characterized by a sharp decrease in glomerular filtrate within days or weeks, being clinically expressed with the inability to excrete nitrogenated waste products and regulate fluid and electrolyte homeostasis.

ARF is one of the most serious problems among renal diseases in the developed world as it entails a high mortality of about 50%. About 30% of all ARF episodes occur in patients admitted to the ICUs as a result of multiple organ failure. In this last context, mortality increases to 80%.

The development of ARF is also one of the most common complications after cardiac interventions amounting to 30,000 a year in Spain and more than 1% of said interventions are conducted in this hospital. Virtually, all the patients that undergo intervention develop certain degree of ARF. The long-term progression of the patients depends on the severity of this post-operation ARF, resulting in mortality close to 60% in those cases that require dialysis after the cardiac intervention (Candela-Toha A, Elias-Martin E, Abraira V et al. Predicting Acute Renal Failure after Cardiac Surgery: External Validation of Two New Clinical Scores. Clin J Am Soc Nephrol; 3:1260-1265. 2008). Both cardiac surgery and kidney transplant are two “quasi” experimental situations for studing ATN in humans since the moment and duration of the ischemic stimulus are known and can also be monitored. All these morbi-mortality statistics have not changed significantly over the last decades and up until now, there is no effective therapy to prevent and/or reduce ATN in all these situations. This is due, in a large extent, to the lack of more precise renal damage markers other than the determination of creatinine and urea in serum used up until now. These conventional markers do not directly reflect cell damage nor do they reflect the compartment of the kidney tissue (tubule or endothelium) in which said damage occurs, they are only parameters indicative of an impaired renal function resulting from damage (Vaidya V S, Waikar S S, Ferguson M A, et al., Urinary Biomarkers for Sensitive and Specific Detection of Acute Renal Injury in Humans. Clin Transl Sci.; 1(3):200-208, 2008). In fact, it is possible that patients with subclinical renal damage are not identified as such because there is no significant alteration in serum creatinine and urea levels. Therefore, over recent years various studies are developed in order to identify and validate new markers for ARF such as NGAL, IL18, KIM, Cystatin C, VEGF or CXCL10, which seem to work as good markers in child populations without extra significant pathologies but not in adult population (Vaidya V S, Waikar S S, Ferguson M A, et al., Urinary Biomarkers for Sensitive and Specific Detection of Acute Renal Injury in Humans. Clin Transl Sci. 1(3):200-208, 2008).

Everything described above justifies the need to identify and validate new, more precise biomarkers for renal damage progression that are indicative of the tissue compartment in which the damage occurs and the degree of the damage and/or recovery, the determination of which is also fast, simple and without a biopsy on the patient being needed.

### DESCRIPTION OF THE INVENTION

Therefore, in a first aspect the present invention relates to a method for obtaining data which can be used for the diagnosis and/or prognosis of acute renal damage which comprises determining the expression level of at least one micro-RNA selected from miR-26b, miR-29a, miR-454, miR-146a, miR-27a, mi-R93, miR-10a in a sample isolated from a subject.

In the present invention, acute renal damage is understood as any damage caused by a sharp decrease in kidney function within hours or days, as a decrease in glomerular filtrate or an accumulation of serum nitrogenated products, or as an inability to regulate homeostasis.

In a second aspect, the present invention relates to a method for the diagnosis and/or prognosis of acute renal damage (hereinafter method of the present invention) which comprises determining the expression level of at least one micro-RNA selected from miR-26b, miR-29a, miR-454, miR-146a, miR-27a, miR-93, miR-10a in a sample isolated from a subject and comparing said expression level with a control value, where the alteration of said expression level is indicative of acute renal damage.

In a more particular embodiment of the present invention, the sample to be analyzed is selected from blood, serum or urine.

In a more particular embodiment of the present invention, the reduction in the serum expression level of miR-26b, miR-29a, miR-454, miR-146a, miR-27a, mi-R93, and/or miR-10a with respect to the control value is indicative of acute renal damage.

In a preferred embodiment of the present invention, the expression of the micro-RNA or micro-RNAs is determined by means of quantitative PCR.

In another preferred embodiment of the present invention, the expression level of the micro-RNA is determined by means of RNA microarrays.

In another preferred embodiment, the method of the present invention comprises determining the expression levels of miR-26b, miR-29a, miR-454, miR-146a, miR-27a, mi-R93 and miR-10a collectively. In another more preferred embodiment, the reduction in the expression level of at least one of the micro-RNAs is indicative of acute renal damage.

In a third aspect, the present invention relates to the use of at least one micro-RNA selected from miR-26b, miR-29a, miR-454, miR-146a, miR-27a, mi-R93 and miR-10a for the diagnosis and prognosis of acute renal damage.

In a fourth aspect, the present invention relates to the use of miR-26b, miR-29a, miR-454, miR-146a, miR-27a, mi-R93 and miR-10a for the diagnosis and prognosis of acute renal damage, collectively.

In a fifth aspect, the present invention relates to a kit for the diagnosis and/or prognosis of acute renal damage (hereinafter kit of the present invention) according to the method of the present invention comprising the probes and primers required for determining the expression level of at least one micro-RNA selected from miR-26b, miR-29a, miR-454, miR-146a, miR-27a, mi-R93 and miR-10a.

In a sixth aspect, the present invention relates to the use the kit of the present invention for the diagnosis and/or prognosis of acute renal damage.

### DESCRIPTION OF THE DRAWINGS

FIG. 1 shows the serum miR-26b expression in ARF patients with ischemic etiology (PI) and toxic etiology (PT) from the time of diagnosis (DO) to 7 days after diagnosis, compared with the miRNA expression in two pools of healthy individuals (healthy). a) Data of amplification cycle in which miRNA expression is detected in all samples (crossing threshold, CT) with respect to a synthetic microRNA used as an internal control of the technique, b) folds of miRNA expression in ARF patients with respect to healthy individuals which are equalized to 1 by means of a exponential mathematical formula.

FIG. 2 shows the serum miR-29a expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

FIG. 3 shows the serum miR-454 expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

FIG. 4 shows the serum miR-146 expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

FIG. 5 shows the serum miR-27a expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

FIG. 6 shows the serum miR-93 expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

FIG. 7 shows the serum miR-10a expression in ARF patients with ischemic etiology (PI) and toxic etiology (PT) from the time of diagnosis (DO) to 7 days after diagnosis, compared with the miRNA expression in two pools of healthy individuals (healthy). a) Data of the amplification cycle in which miRNA expression is detected in all samples (crossing threshold, CT), with respect to a synthetic microRNA used as an internal control of the technique, b) folds of miRNA expression in ARF patients with respect to healthy individuals which are equalized to 1 by means of a exponential mathematical formula.

### DETAILED DESCRIPTION OF THE INVENTION

An experiment for large scale microRNA study was first conducted using RNA obtained from acute renal failure patients with different etiologies. The samples used for this method consisted of:
Two samples from acute renal failure patients with ischemic etiology at the time of maximum renal damage indicated by means of common clinical diagnosis parameters, such as serum creatinine.
Two samples from these same patients 7 or 10 days after renal failure, when the organ function recovered, again estimated by means of serum creatinine.
Two samples from a patient with renal failure caused by nephrotoxic substances, one at the time of maximum damage and the other when renal function recovered.
A sample from a patient having renal failure caused by sepsis, taken at the time of maximum organ damage.
Two groups consisting of 5 healthy people that are used as control.

The experiment for large scale serum microRNA study was conducted by means of quantitative PCR using the Taqman Low Density Array (TLDAs) platforms of Applied Biosystems. By analyzing the data obtained, the microRNAs of interest were selected for study.

The expression data of the selected microRNAs obtained in the previous experiment was subsequently confirmed. To that end, samples from patients other than those mentioned above were used and they consist of:
Four acute renal failure patients with ischemic etiology, referred to as PI1, PI2, PI3, PI4. From each patient, samples on day 0, the time of diagnosis, 1, 3, 5 and 7 days after the diagnosis of renal failure were used.
Three patients having acute renal failure caused by nephrotoxic substances, referred to as PT1, PT2, PT3. From each patient, samples on day 0, the time of diagnosis, 1, 3, 5 and 7 days after the diagnosis of renal failure were used.
Two control groups, each consisting of 10 healthy people.

To confirm the data, quantitative PCR was performed using individual probes for each microRNA of LNA technology (Exiqon).

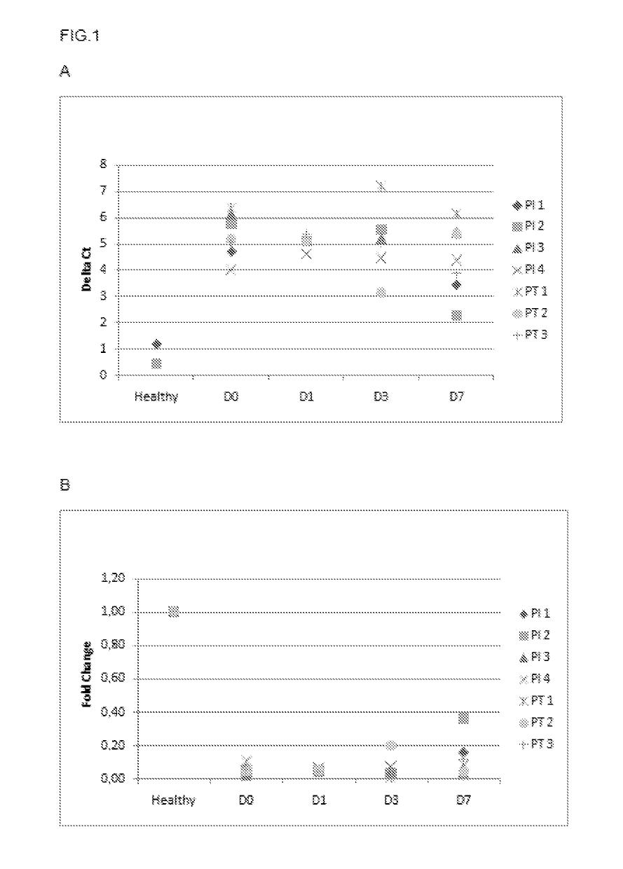

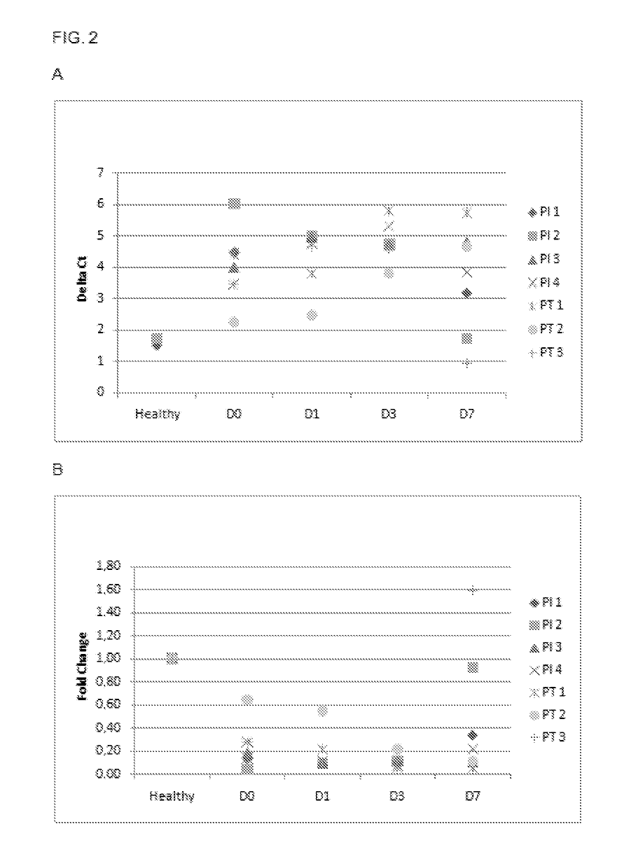

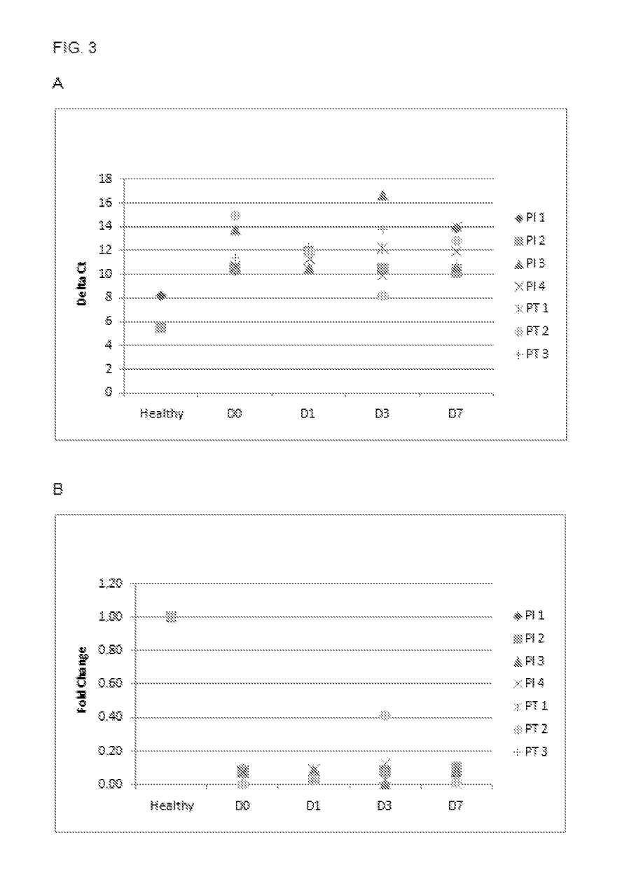

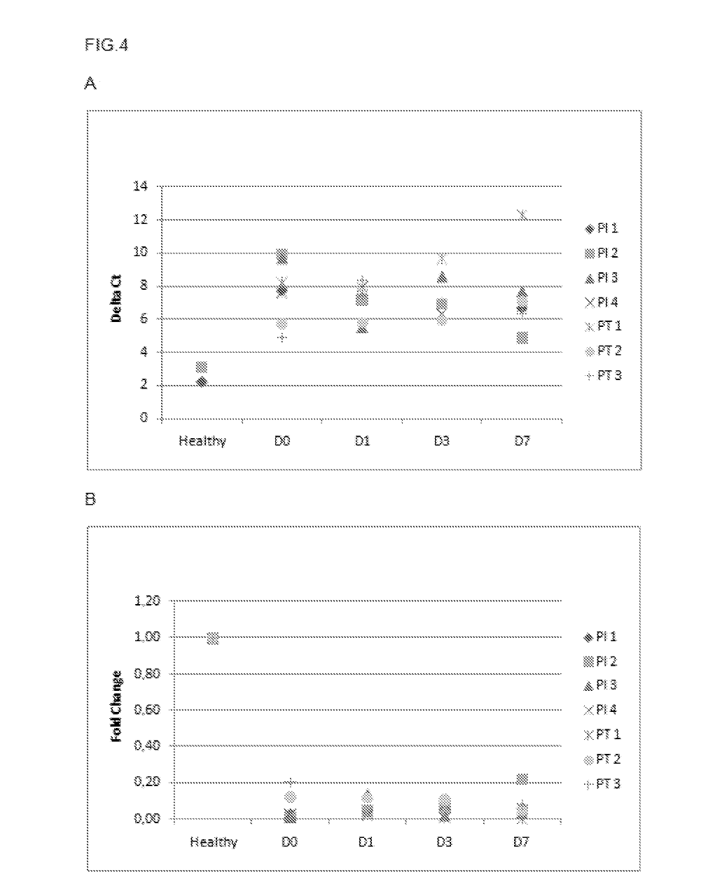

As shown in FIGS. 1-4, serum miRNA expression decreases in ARF patients with respect to healthy controls.

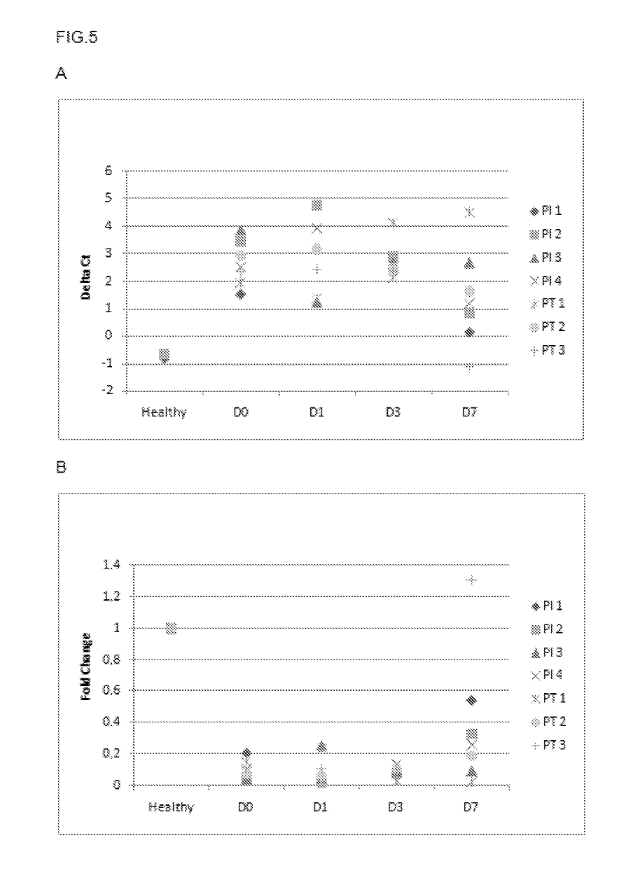

FIG. 5 shows that miR-27a expression decreases in ARF patients with respect to healthy controls. It is important to note that miR-27a expression within 7 days was more variable among patients and some of them had a tendency to recover expression values of healthy individuals.

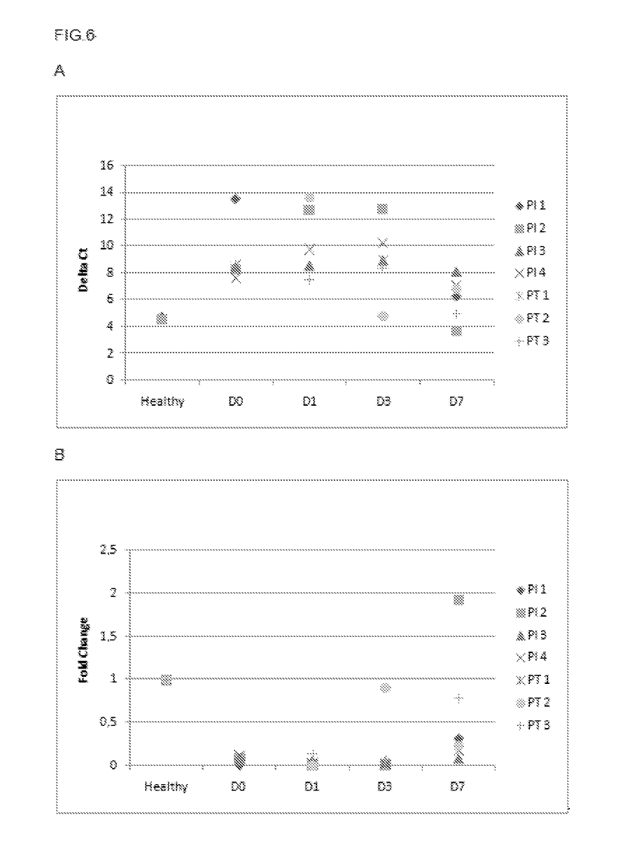

miR-93 expression (FIG. 6) decreased in ARF patients with respect to healthy controls, and it should be also noted that on day 7 this miRNA showed a tendency to recover values that are close to the controls in some patients.

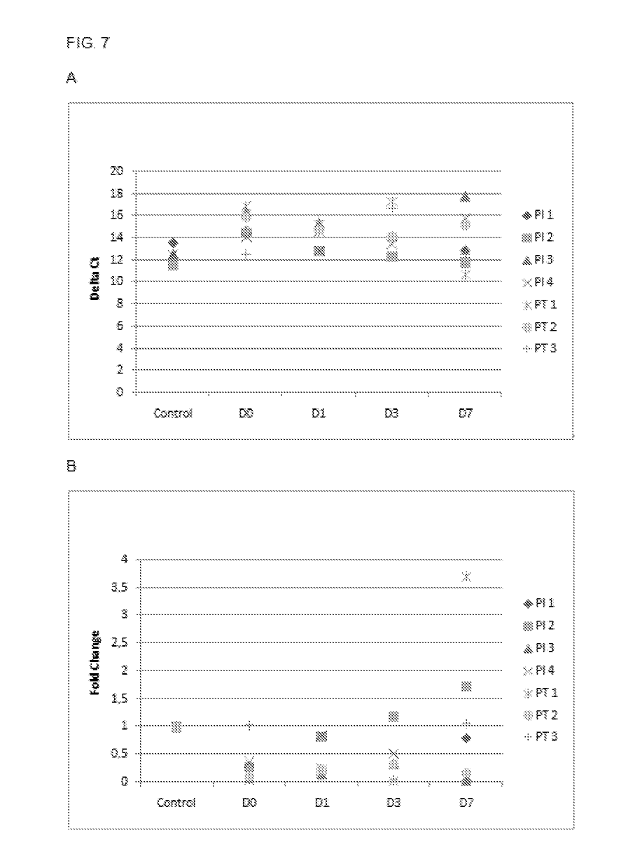

miR-10a expression (FIG. 7) decreased in some ARF patients with respect to healthy controls, this tendency was not observed in an ischemic patient and it is indeed important to note that on day 7 the miRNA expression levels recovered in several patients.

Furthermore, the area under the curve values were calculated by ROC curve analysis for miRNA expression in different patients.

Table 1 shows that these miRNAs had an ARF diagnosis value regardless of the etiology of the ARF, with specificity and sensitivity much greater than serum creatinine (marker used today).

TABLE 1
ROC curve analysis for miRNAs in ARF patients in ICU on day 0

| | | | | 95% Asymptotic | |
|---|---|---|---|---|---|
| | | | | Confidence | |
| Contrast | | | | Interval | |
| result | | Standard | Asymptotic | Lower | Upper |
| variables | Area | error ^{a} | Sig. ^{b} | limit | limit |
| miR_101_D0 | 1.000 | 0.000 | 0.000 | 1.000 | 1.000 |
| miR_210_D0 | 0.761 | 0.120 | 0.043 | 0.526 | 0.995 |
| miR_126_D0 | 0.900 | 0.061 | 0.002 | 0.780 | 1.020 |
| miR_26b_D0 | 1.000 | 0.000 | 0.000 | 1.000 | 1.000 |
| miR_29a_D0 | 0.757 | 0.095 | 0.046 | 0.572 | 0.943 |
| miR_146a_D0 | 1.000 | 0.000 | 0.000 | 1.000 | 1.000 |
| miR_27a_D0 | 0.786 | 0.091 | 0.027 | 0.608 | 0.964 |
| miR_93_D0 | 0.771 | 0.099 | 0.036 | 0.577 | 0.965 |
| miR_10a_D0 | 1.000 | 0.000 | 0.000 | 1.000 | 1.000 |
| miR_127_D0 | 0.714 | 0.122 | 0.097 | 0.474 | 0.954 |

Data of Table 2 demonstrates that these miRNAs were more sensitive and specific than creatinine, suggesting that despite the normal creatinine values, kidney impairment persisted, such that these miRNAs had a high prognosis value with respect to the progression of these patients over time into long-term chronic renal impairment.

TABLE 2
ROC curve analysis for miRNAs in ARF patients in ICU on day 7.

| | | | | 95% Asymptotic | |
|---|---|---|---|---|---|
| | | | | Confidence | |
| Contrast | | | | Interval | |
| result | | Standard | Asymptotic | Lower | Upper |
| variables | Area | Error ^{a} | Sig. ^{b} | limit | limit |
| miR_101_D7 | 0.886 | 0.107 | 0.003 | 0.676 | 1.096 |
| miR_210_D7 | 0.687 | 0.133 | 0.407 | 0.346 | 0.869 |
| miR_126_D7 | 0.729 | 0.106 | 0.077 | 0.520 | 0.937 |
| miR_26b_D7 | 0.986 | 0.019 | 0.000 | 0.949 | 1.022 |
| miR_29a_D7 | 0.625 | 0.137 | 0.333 | 0.357 | 0.893 |
| miR_146a_D7 | 1.000 | 0.000 | 0.000 | 1.000 | 1.000 |
| miR_27a_D7 | 0.571 | 0.118 | 0.580 | 0.339 | 0.804 |
| miR_93_D7 | 0.364 | 0.136 | 0.293 | 0.094 | 0.634 |
| miR_10a_D7 | 1.000 | 0.000 | 0.000 | 1.000 | 1.000 |
| miR_127_D7 | 0.829 | 0.104 | 0.319 | 0.424 | 0.833 |

Data of Table 3 shows that the alteration in these miRNAs indicated a predisposition to developing ischemic ARF after cardiac surgery.

TABLE 3
ROC curve analysis for miRNAs in patients after cardiac surgery.

| | | | | 95% Asymptotic | |
|---|---|---|---|---|---|
| | | | | Confidence | |
| Contrast | | | | Interval | |
| result | | Standard | Asymptotic | Lower | Upper |
| variables | Area | Error ^{a} | Sig. ^{b} | limit | limit |
| miR101_b | 0.899 | 0.065 | 0.001 | 0.772 | 1.026 |
| miR210_b | 0.702 | 0.112 | 0.082 | 0.482 | 0.911 |
| miR126_b | 0.848 | 0.075 | 0.003 | 0.701 | 0.996 |
| miR26b_b | 0.944 | 0.039 | 0.000 | 0.869 | 1.020 |
| miR29a_b | 0.803 | 0.089 | 0.009 | 0.629 | 0.977 |
| miR146a_b | 0.778 | 0.087 | 0.017 | 0.608 | 0.947 |
| miR27a_b | 0.896 | 0.064 | 0.001 | 0.771 | 1.021 |
| miR93_b | 0.934 | 0.049 | 0.000 | 0.838 | 1.301 |
| miR10a_b | 0.697 | 0.127 | 0.090 | 0.448 | 0.946 |
| miR127_b | 0.859 | 0.068 | 0.002 | 0.726 | 0.991 |

Data of Table 4 shows that the alteration in these miRNAs are early indicators of ARF development after cardiac surgery.

TABLE 4
ROC curve analysis for miRNAs in patients immediately
after cardiac surgery.

| | | | | 95% Asymptotic | |
|---|---|---|---|---|---|
| | | | | Confidence | |
| Contrast | | | | Interval | |
| result | | Standard | Asymptotic | Lower | Upper |
| variables | Area | Error ^{a} | Sig. ^{b} | limit | limit |
| miR101 pi | 0.667 | 0.118 | 0.154 | 0.434 | 0.899 |
| miR210_pi | 0.598 | 0.119 | 0.402 | 0.365 | 0.831 |
| miR126_pi | 0.667 | 0.119 | 0.154 | 0.434 | 0.899 |
| miR26b_pi | 0.757 | 0.101 | 0.028 | 0.558 | 1.955 |
| miR29a_pi | 0.561 | 0.110 | 0.603 | 0.344 | 0.777 |
| miR146a_pi | 0.534 | 0.120 | 0.769 | 0.299 | 0.770 |
| miR27a_pi | 0.831 | 0.091 | 0.005 | 0.652 | 1.009 |
| miR93_pi | 0.852 | 0.078 | 0.003 | 0.699 | 1.004 |
| miR10a_pi | 0.836 | 0.098 | 0.004 | 0.644 | 1.028 |
| miR127_pi | 0.751 | 0.092 | 0.032 | 0.570 | 0.932 |

1-12. (canceled)

13. A method of measuring the expression level of miR-26b in a subject, said method comprising:
a. obtaining at least one serum sample from a subject suspected of having acute renal damage or being at an increased risk of having acute renal damage; and
b. measuring the expression level of miR-26b in the sample by contacting the sample with a PCR primer specific for miR-26b and amplifying miR-26b.

14. The method of claim 13, wherein step b further comprises measuring the expression level of at least one additional miRNA selected from the group consisting of: miR-29a, miR-454, miR-146a, miR-27a, miR-93 and miR-10a in the sample.

15. The method of claim 14, wherein measuring of the expression level of the at least one additional miRNA comprises contacting the sample with a PCR primer specific for the at least one additional miRNA and amplifying the at least one additional miRNA.

16. The method of claim 13, wherein step b further comprises measuring the expression level of miR-29a, miR-454, miR-146a, miR-27a, miR-93 and miR-10a in the sample.

17. The method of claim 16, wherein measuring of the expression level of miR-29a, miR-454, miR-146a, miR-27a, miR-93 and miR-10a comprises contacting the sample with PCR primers specific for miR-29a, miR-454, miR-146a, miR-27a, miR-93 and miR-10a and amplifying miR-29a, miR-454, miR-146a, miR-27a, miR-93 and miR-10a.
